(19) United States
(12) Patent Application Publication
(10) Pub. No.: US 2010/0104547 A1
(43) Pub. Date: Apr. 29, 2010

(54) TOPICAL COMPOSITION FOR THE TREATMENT OF ALLERGENIC EFFECTS

(76) Inventor: Lawrence M. Logsdon, Laguna Niguel, CA (US)

Correspondence Address: STETINA BRUNDA GARRED & BRUCKER, 75 ENTERPRISE, SUITE 250, ALISO VIEJO, CA, 92656, US

(21) Appl. No.: 12/683,388

(22) Filed: Jan. 6, 2010

Related U.S. Application Data
(63) Continuation of application No. 11/270,924, filed on Nov. 11, 2005.

(51) Int. Cl. A61K 38/48; A61P 17/00

(52) U.S. Cl. 424/94.3

(57) ABSTRACT

A topical composition for treating stings and insect bites effectively significantly reducing pain and/or eliminating it (wiping away pain, or WAP) is disclosed comprising a stabilized proteonaise, antibacterial/anti-microbial, a cooling agent, an anti-inflammatory agent, a debriding agent and an exfoliating agent.

### I. BACKGROUND OF THE INVENTION

This invention is directed to non-prescription topical compositions for treatment of allergenic and other deleterious effects such as itching, pain and swelling resulting from insect stings and bites, in addition to areas of skin contact with noxious plants.

It is known to employ methods for reducing the itch associated with the bite of a blood-feeding insect, such as a mosquito or black fly, which involves applying a topical pharmaceutical formulation of papain, pancreatin or subtilisin to the skin proximate to the bite. Adult black flies, notably Simulium vernustum and Prosimulium hirtipes, and mosquitoes of the Aedes, Anopheles and Culex genera are well known for their blood feeding habit. The feeding behavior involves puncturing the skin of the host, injecting a small quantity of anticoagulant, and ingesting blood. The reaction of the human host to the injection of the anticoagulant gives rise to swelling, redness and itching in the area immediately surrounding the bite.

Numerous preparations are presently available to provide relief from the allergenic effects of insect stings and bites or for the relief from the effects of contact with the skin of noxious plants. Most of these preparations are effective only to a degree and merely tend to anesthetize the site of the affected area and do little to draw the actual material causing the allergenic reaction from the site.

Historically, Indians in Central America employed arrowroot (a starch taken from the root of a plant) for the treatment of poison arrow wounds which gave rise to the name. Presently arrowroot is used as a starch in cooking and appears to have no significant medicinal uses.

Prior art methods include relieving the allergenic effects of insect stings, insect bites and areas of skin contact with noxious plants by applying topically to the affected area a composition containing at least either arrowroot, a calcium phosphate, or a combination of them both as an active absorbent ingredient.

There have been anecdotal reports of the utility of papain, commonly in the form of meat tenderizer, for the first aid treatment of hymenopteria stings. It has been suggested that a paste material can be applied diminishing the swelling resulting from the sting. However, it is acknowledged that the efficacy of papain in reducing bee venom toxicity suggested in the anecdotal reports has not been confirmed in the known literature. In Agostinucci et al [Toxicon 19, 851-855 (1981)] the effect of papain paste which was topically applied after an intradermal injection of bee venom in mice concluded that the results do not substantiate the theory. There was no marked inhibition of lesion development apparent in any of the animals receiving papain or Adolph's Meat Tenderizer by intradermal injection or topical application.

Contrasting the inefficacy of papain in the treatment of stings by venomous insects, it is to be noted that papain, pancreatin and subtilisin are effective in reducing the itch associated with the bites of blood feeding insects. It is postulated that the efficacy as demonstrated in one case and not in the other may arise from a distinction in the etiology of the two reactions. For example, in the case of stinging insects, a highly specialized mixture of irritants and lytic agents is injected from a specially evolved gland; the venom is particularly adapted to cause tissue destruction, pain and/or paralysis. On the other hand, the material injected by biting insects (mosquitoes and black flies) is essentially saliva modified to inhibit blood coagulation. In any event, it has been found that the topical application of papain, pancreatin or subtilisin is effective in reducing the itch associated with mosquito and black fly bites.

In U. S. Pat. No. 4,444,751 there is disclosed an anti-venom composition and, particularly, a topical preparation for the treatment of noxious stings of plant and animal origin employing proteolytic enzymes and, in particular, papainase which are stated to be effective in neutralizing toxicants introduced into the epidermis by stings, bites or the like.

In U. S. Pat. No. 5,543,149 there is disclosed a method and composition for reducing the itch associated with insect bites employing a therapeutically effective amount of papain, pancreatin or subtilisin in a suitable pharmaceutical carrier to the surface of the skin proximate to the bite.

In U. S. Pat. No. 4,083,965 there is disclosed a method of relieving the allergenic effects of insect stings, insect bites and areas of skin contact with noxious plants comprising applying topically to the affected area a composition containing at least either arrowroot, a calcium phosphate or a combination of them both as an active absorbent ingredient.

In U. S. Pat. No. 5,670,142 there is disclosed a method and composition for reducing the itching associated with chicken pox comprising applying a therapeutically effective amount of papain, pancreatin or subtilisin or a combination thereof in a suitable pharmaceutical carrier to the surface of the skin proximate to the varicella vesicles.

In U. S. Pat. No. 5,441,740 there is disclosed compositions comprising alpha hydroxyacids, salicylic acid and digestive enzymes which are stated to be effective when topically applied to prevent as well as alleviate various skin conditions including wrinkles and dry skin.

In U. S. Pat. No. 3,860,702 there is disclosed the use of proteolytic enzymes which can be absorbed through the dermis and epidermis if they are carried in a carrier made up of partially hydrolized cocoa butter to provide anti-inflammatory compositions and particularly those compositions which are applied topically for the treatment of inflammations in the subdermal tissue.

In U. S. Pat. No. 5,849,729 there is disclosed a cocoa butter composition employed with partially hydrolyzed cocoa butter emulsion as a means for facilitating the absorption of medicaments through the skin to provide control of epidermal, derma and transdermal penetration of various topically applied pharmacologically active agents.

In U. S. Pat. No. 4,678,668 there is disclosed a composition and method for reducing soft tissue swelling and pain in humans and animals by topically administering a therapeutic composition comprising a topically administerable transfer vehicle and a proteolytic enzyme.

Other compositions are available including after sting gel, bee and wasp compositions employing ammonia, and various other compositions to treat bee venom, swelling, burning pain and redness for the resulting swelling whether caused by bees, insect bites, hives or other irritants under such designations as Apis mellifica (honeybee).

Although the prior art is replete with topical compositions for treating humans and other animals for bites and stings, particularly bites of black flies, mosquitoes, jelly fish and bees, there is still a demonstrated need for an effective composition which is stable, pain relieving, antimicrobial and promotes deep penetration of the active ingredients and a mild abrasion factor to debride and open up the wound areas.

### II. OBJECTS OF THE INVENTION

It is therefore an object of this invention to provide a topical composition for treating humans and other animals for bites and stings devoid of the above noted deficiencies.

It is a further object of this invention to provide a topical composition for treating stings and insect bites providing an effective cooling agent.

A further object of this invention is to provide a topical composition for treating stings and insect bites which provides the requisite pain relief or exfoliating agent.

Yet another object of this invention is to provide a topical composition for treating stings and insect bites which provides anti-inflammatory properties.

Yet again another object of this invention is to provide a topical composition for treating stings and insect bites which includes a driving factor to promote deep penetration of the active ingredients.

Still another object of this invention is to provide a topical composition for treating stings and insect bites which provides a mild abrasion factor to debride and open up the wound areas for more effective use of the topical composition.

### III. SUMMARY OF THE INVENTION

These and other objects of the instant invention are accomplished generally speaking by providing a topical composition for treating stings and insect bites which effectively wipes away pain, hereinafter referred to as a WAP composition, comprising a stabilized proteonaise, an antibacterial/anti-microbial, a cooling agent, an anti-inflammatory agent, a debriding agent and an exfoliating agent.

In a preferred embodiment, the composition may also include a driving factor which in addition inhibits the formation of bacteria in the form of a non-ancreous gel base. To further enhance this composition, an emollient which also acts as a preservative may also be employed to preserve the integrity of the composition and provide further therapeutic benefits.

Although any suitable antibacterial/anti-microbial agent may be employed in the composition of the instant invention, it is preferred to employ mella luca sitrifolia, commonly referred to as tea tree oil.

Any suitable stabilized proteonaise enzyme may be employed in the composition of the instant invention. Typical proteonaise enzymes include papain, bromelain, pepsin or other peptide-bond breaking enzymes. Preferred of these is stabilized pepsin enzyme.

The stabilized pepsin of the instant invention is provided by screening pepsin granules through a 40 mesh screen to provide granules of 40 mesh or greater. These granules are then coated with a thin layer of paraffin wax employing conventional techniques on a tray and allowed to dry. The resulting stabilized pepsin enzyme is surprisingly found to have a shelf life of five times typical unstabilized enzymes, for example, typically the shelf life of pepsin resulting from this stabilization process has a shelf life of five years as compared to unstabilized enzymes which typically have shelf lives of about one year.

Any suitable cooling agent may be employed in the composition of the instant invention. Typical cooling agents include menthol and camphor and mixtures thereof. It is preferred to employ mixtures of menthol and camphor which, in addition to providing a cooling agent effect, also act as anti-inflammatories. Any suitable anti-inflammatory agent may be employed in the composition of the instant invention. Typical anti-inflammatories include vitamin C, benadryl, aspirin. Preferred of these are menthol and camphor.

Any suitable nonaqueous gel base may be employed in the composition of the instant invention. Typical such bases include propylene glycol glycerin and alcohol typically combined with a gelling agent and a thickening agent such as sodium sarcosinate. Preferred of these is propylene glycol and a conglomerate known in the industry as seppy gel 305 with a thickening agent sodium sarcosinate.

Any suitable debriding agent may be used in the composition of the instant invention. Typical debriding agents include walnut shells, abrasive particulate matter, such as polypropylene beads. Preferred of these in the composition of the instant invention are ground walnut shells.

Any suitable exfoliating agent may be used in the composition of the instant invention. Typical exfoliating agents include polyethylene beads and walnut shells. Preferred of these in the composition of the instant invention are a combination of polyethylene beads and ground walnut shells.

Any suitable preservative and emollient may be used in the composition of the instant invention. Preferred of these as a preservative in the composition of the instant invention is vitamin E dl alpha tocopherol. Preferred as an emollient is propylene glycol in the composition of the instant invention.

The stabilized pepsin as employed in the composition of the instant invention may be employed in any suitable proportion or weight ratio. Typically, the stabilized pepsin enzyme is employed in a weight ratio of from 1-4%. Preferably, the pepsin is present in a weight percentage of 2.5%. The other substituents of the WAP composition of the instant invention are present in weight ratio as follows:

Propylene glycol—about 80%;

Menthol—about 3%;

Camphor—about 4%;

the binder or conglomerate—about 7%;

the thickening agent—about 1%;

the debriding agent—about 4%;

the exfoliating agent—about 4%; and

the emollient and preservative—about 0.25%

While the present invention has been particularly described with respect to preferred compositions of preferred substituents, it will be understood that the invention is not limited to these particular compositions and substituents described in the preferred embodiments. For example, 350 pounds of propylene glycol is added to a 100-gallon stainless steel main tank; 20.85 pounds of menthol and 33 pounds of camphor are mixed in a separate stainless steel tank; and the menthol/camphor mixture is added slowly to the main tank into the propylene glycol. 9.45 grams of vitamin E dl alpha tocopherol is then slowly added and dissolved into the main tank mixture with continuous mixing. 50 pounds of propylene glycol is then added to a separate steel mixing tank to which 20.85 pounds of stabilized pepsin resin is added which dissolves during continuous mixing. This mixture is then added to the main tank employing continuous mixing. 56.23 pounds of seppy gel 305 is then slowly added to effect dissolution into the main tank mixture. Then, 8.33 pounds of sodium sarcosinate hamposyl L-95 is slowly added to dissolve into the main tank mixture employing continuous mixing. 33.32 pounds of walnut shells (texshel) is then added to the main tank employing continuous mixing. 33.32 pounds of A.C. polymer polyethylene beads are then added to the main tank employing continuous mixing. Mixing is performed until a uniform and homogenous solution is obtained. The mixture in the main tank is continuously mixed to obtain a uniform mixture with a smooth texture free from lumps.

While the present invention has been particularly described with respect to certain substituent elements in its preferred embodiment, it will be understood that the invention is not limited to these particular substituents described in the preferred embodiments, or the sequence in adding substituents or processing the substituents. On the contrary, it is intended to cover all alternatives, modifications, and equivalents as may be included within the spirit and scope of the invention defined by the appended claims.

In addition, other substituents may be employed in the compositions of the instant invention as claimed as well as variations and alternatives to the methods disclosed and claimed with similar results with regard to the antibiotic, anti-microbial and anesthetic characteristics of these compositions. In particular, the scope of the invention is intended to include, for example, those substituents which further enhance the solubility and appearance of these compositions, in addition to other substituents which provide further synergy to these herbal compositions.

1. A topical composition for significantly reducing or eliminating pain associated with stings and insect bites, comprising a stabilized proteinase, a cooling agent, an antibacterial agent mella luca sitrifolia, an anti-inflammatory agent, a debriding agent and an exfoliating agent.

2-21. (canceled)

22. The topical composition as defined in claim 1 wherein said proteinase enzyme is selected from the group consisting of papain, bromelain, pepsin and other peptide-bond breaking enzymes.

23. The composition as defined in claim 1 wherein said proteinase enzyme is stabilized pepsin enzyme.

24. The composition as defined in claim 1 wherein said cooling agent is selected from the group consisting of menthol and camphor and mixtures thereof.

25. The composition as defined in claim 1 wherein said anti-inflammatory agent is selected from the group consisting of vitamin C, Benadryl, aspirin, and combinations thereof.

26. The composition as defined in claim 1 wherein said debriding agent is selected from the group consisting of walnut shells, polypropylene beads, and combinations thereof.

27. The composition as defined in claim 1 wherein said exfoliating agent is selected from the group consisting of walnut shells, polyethylene beads, and combinations thereof.

28. The composition as defined in claim 1 further comprising a preservative and emollient agent.

29. The composition as defined in claim 28 wherein said preservative and emollient agent is vitamin E dl tocopherol.

30. A topical composition for treating stings and insect bites comprising a stabilized proteinase, pain relieving agent, a cooling agent, an antibacterial/anti-microbial agent mella luca sitrifolia, an anti-inflammatory agent, a debriding agent, and exfoliating agent and further comprising a non-aqueous gel base which inhibits bacteria.

31. The composition as defined in claim 30 further comprising a gel base which binds the composition together.

32. The composition as defined in claim 30 further comprising a thickening agent.

33. The composition as defined in claim 30 wherein said non-aqueous gel base which inhibits bacteria comprises propylene glycol and mella luca sitrifolia.

34. The composition as defined in claim 30 wherein said thickening agent comprises sodium sarcosinate.

35. A topical composition for treating stings and insect bites comprising pepsin present by weight at about 2½%, propylene glycol present by weight at about 80%, menthol present by weight at about 3%, camphor present by weight at about 4%.

36. The composition as defined in claim 35 further comprising a binder or conglomerate present by weight at about 7%.

37. The composition as defined in claim 35 further comprising sodium sarcosinate present by weight at about 1%.

38. The composition as defined in claim 35 further comprising a debriding agent from the group consisting of walnut shells, polypropylene beads, or a combination thereof present by weight at about 4%.

39. The composition as defined in claim 35 further comprising an exfoliating agent selected from the group consisting of walnuts shells, polyethylene beads, and combinations thereof present by weight at about 4%.

40. The composition as defined in claim 35 further comprising vitamin E dl tocopherol present by weight at about 0.25%.
